(19) United States
(12) Patent Application Publication
(10) Pub. No.: US 2008/0311667 A1
(43) Pub. Date: Dec. 18, 2008

(54) Exogenous Markers for Oxidative Stress

(76) Inventors: Jacob Vaya, Merom Hagalil (IL); Snait Tamir, Higher Galilee (IL)

Correspondence Address: BROWDY AND NEIMARK, P.L.L.C.;624 NINTH STREET, NW, SUITE 300, WASHINGTON, DC, 20001-5303, US

(21) Appl. No.: 11/910,405

(22) PCT Filed: Mar. 30, 2006
(86) PCT No.: PCT/IL06/00409

§ 371 Date: Jul. 15, 2008

Related U.S. Application Data
(60) Provisional application No. 60/666,171, filed on Mar. 30, 2005.

(51) Int. Cl. G01N 33/92; G01N 33/50; C07H 19/16; C07J 1/00

(52) U.S. Cl. 436/71; 436/94; 536/27.21; 552/502

(57) ABSTRACT

The invention provides oxogenous markers, designed and synthesized for the measurement and characterization of oxidative/nitrosative stress levels, thus enable the identification of the type of reactive ROS/NRS involved, characterization of the damaged products and their formation kinetics, and thereby the identification of pathological conditions associated with oxidative/nitrosative stress, before appearing or at the stage of development.

### FIELD OF THE INVENTION

The present invention relates to exogenous markers for oxidative stress, their preparation and uses thereof.

Abbreviations: BHT: butylated hydroxytoluene; BSA: N,O-bis(trimethylsilyl) acetamide; DAD: diode-array detector; DCM: dichloromethane; DDW: double-distilled water; DMEM: Dulbecco's Modified Eagle Medium; DMF: dimethylformamide; EDC: ethylene dichloride; EDTA: ethylenediaminetetraacetic acid; FCS: fetal calf serum; FTIR: Fourier Transform Infra-Red (spectroscopy); HBSS: Hank's balanced salt solution; HPLC: high performance liquid chromatography; LA: linoleic acid; LC: liquid chromatography; MPM: mouse peritoneal macrophages; MS: mass spectrometry; NBS: N-bromosuccinimide; PBS: phosphate-buffered saline; ph₃P: triphenylphosphine; PMSF: phenylmethanesulfonyl fluoride; PTSA: p-toluenesulfonic acid; PUFA: polyunsaturated fatty acid; RNS: reactive nitrogen species; ROS: reactive oxygen species; SIN-1: 3-morpholinosydnonimine; TBAF: tetrabutylammonium fluoride hydrate; TCL: N-linoleyl tyrosine cholesteryl ester; TGL: N-linoleyl tyrosine 2′-deoxyguanosine ester; TL: N-linoleyl tyrosine; Ty: tyrosine.

### BACKGROUND OF THE INVENTION

Oxidative stress is known to be involved in several human diseases such as cardiovascular diseases, cancer, inflammation-related diseases and aging (Finlcel and Holbrook, 2000; Halliwell, 2002; Aviram, 2000). Reactive oxygen and nitrogen species (ROS/RNO) such as superoxide (O₂⁻.), hydrogen peroxide (H₂O₂), hydroxyl radical (HO.), nitrogen oxide (NO.), peroxynitrite (ONOO⁻) and hypochlorous acid (HOCl), are all products of normal metabolic pathways in humans which may either be useful, e.g., in the destruction of invading organisms, or cause unwanted collateral damage to normal neighboring cells (Saran et al., 1999). ROS/RNS also take part in signaling cascades and are involved in cellular functions such as cell proliferation, inflammation and adhesion. Under certain conditions, ROS/RNS exert harmful effects (Halliwell and Gutteridge, 1999). RNS derived from NO can be strong oxidants, which can oxidize lipids, proteins and DNA and formed in vivo under pathological conditions such as inflammation (Eiserich et al., 1998) and atherosclerosis (Beckmann et al., 1994).

Organs protect themselves from the toxicity of excess ROS by maintaining an oxido/redox balance in different ways, including the use of endogenous and exogenous antioxidants. An excess of ROS is termed oxidative stress, and is associated with changes in the structure and function of such biomolecules as DNA, proteins and lipids, which may lead to the development of cardiovascular diseases, diabetes, neurodegenerative disorders, cataract, cancer and aging. Reactions of ROS with DNA modify its bases and lead to the development of mutations.

Cells and tissues contain polyunsaturated fatty acids (PUFA), mainly linoleic acid and arachidonic acid, as esters with glycerol (phospholipids) and cholesterol. These PUFA are readily auto-oxidized under oxidative stress and, due to their double bonds, react with ROS, thereby initiating other free radical reactions. Auto-oxidation of PUFA affects cell function and generates changes in membrane density, fluidity, permeability and oxido/redox potential.

The identification of reliable biomarkers, e.g. modified endogenous compounds formed in biological systems as a result of oxidative stress, is essential for the prediction of the early development of pathological conditions (Aviram and Vaya, 2001). Several modified endogenous compounds have been proposed as indicators of oxidative stress to proteins (chloro- or nitro-tyrosine) and lipids (oxidized degradation products of PUFA and oxysterols). The detection of these biomarkers as indicators of oxidative stress suffers from limitations as a result of insufficient specificity, accuracy and reliability. Levels of such biomarkers are determined by a dynamic process involving their formation, accumulation and removal, which may not be due to the oxidative process within the system, but rather to alternative processes. Furthermore, their levels may change during their isolation and sample preparation and their application in vivo is questionable (Halliwell, 2002).

We have recently shown that cholesterol, linoleic acid and/or tyrosine yield different spectra of products when oxidized, alone or in a mixture of the three, by various oxidative systems (Szuchman et al., 2003).

Due to the limitations of the prior art, it would be of great importance to provide specific and reliable markers of oxidative stress.

### SUMMARY OF THE INVENTION

The present invention is based on a new concept whereby an exogenous marker is designed and synthesized for the measurement and characterization of oxidative stress levels. According to the present invention, this new concept enables the identification of the type of relative ROS/NRS involved, characterization of the damaged products and their formation kinetics, and thereby the identification of pathological conditions associated with oxidative stress either existing, before appearing or at the stage of development in the individual under examination.

In one embodiment, the designed marker is composed of tyrosine (Ty) and linoleic acid (LA) attached together covalently through an amide bond forming the N-linoleyl tyrosine molecule (herein designated “TL”). In the TL molecule, the active sites, i.e. the aromatic ring of Ty and the two double bonds of LA remain intact. In addition, the strong amide bond connecting the tyrosine and the linoleic acid is not likely to be easily hydrolyzed chemically or enzymatically by proteases.

In another embodiment, the designed marker is composed of tyrosine (Ty), linoleic acid (LA) and either cholesterol or 2′-deoxyguanosine, in which the Ty and LA residues are attached covalently through an amide bond, the cholesterol residue is attached covalently through an ester bond to the carboxyl group of the tyrosine and the 2′-deoxyguanosine residue is attached covalently through the 6′ OH position to the carboxyl group of the tyrosine (herein designated “TCL” and “TGL” respectively).

The compounds of the invention are useful as exogenous markers of oxidative stress.

The present invention thus relates to a method of assessing oxidative stress which comprises incubation of a biological sample with the oxogenous marker of the present invention, whereby an increase of the amount of at least one oxidation product of said marker in the biological sample compared to a control sample indicates oxidative stress.

The biological sample according to the present invention is blood, urine or saliva sample.

The present invention also relates to the use of the TL, TCL and TGL markers for early identification of diseases, disorders and pathological conditions associated with oxidative/nitrosative stress, close to or before the onset of the disease, disorder or pathological condition.

The present invention further relates to the use of the markers TL, TCL and TGL for monitoring the efficacy of a treatment of a disease, disorder or pathological condition associated with oxidative/nitrosative stress, by measurement of the oxidated products before, during and after the treatment.

### BRIEF DESCRIPTION OF THE FIGURES

FIG. 1 shows the oxidized products from HOCl-induced N-linoleyl tyrosine (TL) oxidation in the cell-free system and in cells. TL (40 μM) in HBSS was incubated with PLB cells for 3 h and then HOCl was added (0.2 mM). After 30 min, the cells were extracted and the organic phase was analyzed by LC/MS/MS. The effect of HOCl on the oxidation of TL in the cell-free system was studied under similar conditions.

FIG. 2 shows the oxidized products from copper ions-induced TL oxidation in a cell-free system and in J774 cells. Cells were incubated with copper ions (20 μM) and TL (40 μM). After 10 h, the cells were extracted and the organic phase was analyzed in LC/MS/MS. The effect of copper ions on the oxidation of tyrosine N-linoleyl in a cell-free system was studied under the same conditions.

FIGS. 3A-3B show the oxidized products from SIN-1-induced TL oxidation in a cell-free system (5A) and in cells (5B). TL (40 μM) in HBSS was incubated with J774 cells for 3 h prior to the addition of SIN-1 (0.1 mM). After 1 h, the cells were extracted and the organic phase was analyzed in LC/MS/MS. The effect of SIN-1 on the oxidation of TL in the cell-free system was studied under similar conditions.

FIG. 4 shows the oxidative effect of stimulated mouse peritoneal macrophages (MPM) from BALB/c versus E⁰ mice on exogenous TL in cells. MPM were harvested from BALB/c and E⁰ mice 3 days after intraperitoneal injection of 3 ml 4% thioglycolate. The cells were washed and incubated for 4 h as described in Materials and Methods. Cells were then incubated with 40 μM or 80 μM TL. After 20 h, the medium was removed and the cells were harvested, extracted and injected into LC/MS.

FIGS. 5A-5B show the oxidized products of HOCl-induced N-linoleyl tyrosine cholesteryl ester (TCL) oxidation in a cell-free system, divided into the oxidized TL products identify either the oxidation of the tyrosine moiety or the linoleic acid moiety (5A); and the oxidized products of the cholesterol moiety (oxysterols) (5B).

FIGS. 6A-6D show the total amount of oxidative products of exogenous TCL as a result of incubation with blood from hypercholesterolemic patients (Hc). After incubating with the TCL, the blood samples were allowed to coagulate at room temperature and the plasma was obtained after centrifugation. Hydrolizing the ester bond of the TCL resulted in Ox-TL and oxysterols which were analyzed separately using LC/MS/MS and GC/MS, respectively. Total Ox-TL vs. total TL in the serum (6A) and in the blood cells (6B); and total oxysterols vs. total cholesterol in the serum (6C) and in the blood cells (6D).

FIGS. 7A-7D show the total amount of oxidative products of exogenous TCL as a result of incubation with blood from diabetic patients. Incubation phase and the analysis were done as described in FIG. 6. Total Ox-TL vs. total TL in the serum (7A) and in the blood cells (7A); and total oxysterols vs. total cholesterol in the serum (7C) and in the blood cells (7D).

FIGS. 8A-8C show the total amount of oxidative products of exogenous TCL as a result of incubation with blood from diabetic patients before and after 1 week of pomegranate juice (PJ) supplementation. Incubation phase and the analysis were done as described in FIG. 6. Total Ox-TL vs. total TL in the serum (8A) and in the blood cells (8B); and total oxysterols vs. total cholesterol in the blood cells (8C).

FIGS. 9A-9D show the oxidized products of exogenous TCL as a result of incubation with blood from Parkinson patients. Incubation phase and the analysis were done as described in FIG. 6. Total Ox-TL vs. total TL in the serum (9A) and in the blood cells (9B); and total oxysterols vs. total cholesterol in the serum (9C) and in the blood cells (9D).

### DETAILED DESCRIPTION OF THE INVENTION

Reactive Oxygen Species (ROS) is a term collectively describing radicals and other non-radical reactive oxygen derivatives. These intermediates may participate in reactions giving rise to free radicals or that are damaging to organic substrates. Reactive Nitrogen Species (RNS) are radical nitrogen-based molecules that can act to facilitate nitrosylation reactions.

Oxidative stress occurs when the generation of ROS in a system exceeds the system's ability to neutralize and eliminate them. If not regulated properly, the excess ROS can damage a cell's lipids, protein or DNA, inhibiting normal function. Because of this, oxidative stress has been implicated in a growing list of human diseases as well as in the aging process.

Nitrosative stress occurs when the generation of RNS in a system exceeds the system's ability to neutralize and eliminate them. Nitrosative stress may lead to nitrosylation reactions that can alter protein structure thus inhibiting normal function.

Oxidative stress and nitrosative stress are known or suspected to play a role in cardiovascular diseases such as atherosclerosis, ischemia/reperfusion injury, restenosis and hypertension; cancer; inflammatory diseases such as acute respiratory distress syndrome (ARDS), asthma, inflammatory bowel disease (IBD), dermal and ocular inflammation and arthritis; metabolic diseases such as diabetes; and diseases of the central nervous system (CNS) such as amyotrophic lateral sclerosis (ALS), Alzheimer's disease, Parkinson's disease, and stroke. The increased awareness of oxidative stress related to disease and the need to measure the delicate balance that exists between free radicals and the systems in place to regulate them has given rise to a demand for new research tools.

The present invention provides exogenous markers of oxidative and nitrosative stress that enables the identification of the type of relative ROS/NOS involved and thereby to identify pathological conditions associated with oxidative stress. In this way, the exogenous markers are tools both for diagnosis of a pathological condition associated with oxidative/nitrosative stress at its early onset or before its onset and for monitoring the treatment of such a pathological condition with specific drugs or other treatments.

The present invention thus relates to a compound of formula I:

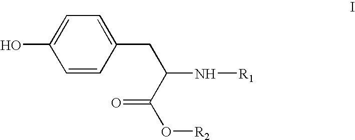

wherein R₁ is linoleyl, and R₂ is H, cholesteryl or a 2′-deoxyguanosine residue.

The compounds of the formula I are useful as markers for oxidative/nitrosative stress and will be referred to herein in the specification interchangeably as “compound(s) of formula I” or as “marker(s)”.

In one embodiment of the invention, the marker is the compound of formula I wherein R₂ is H, namely, the compound N-linoleyl tyrosine, herein identified as TL or compound 4.

In another embodiment of the invention, the marker is the compound of formula I wherein R₂ is cholesteryl, namely, the compound N-linoleyl tyrosine cholesteryl ester, herein identified as TCL or compound 7.

In a further embodiment of the invention, the marker is the compound of formula I wherein R₂ is a 2′-deoxy-guanosine residue attached through the 6′ OH position, namely, the compound N-linoleyl tyrosine 2′-deoxyguanosine ester, herein identified as TGL or compound 8.

The compounds of formula I of the invention can be prepared by standard synthetical methods as well known in the art. For example, they can be prepared by the method depicted in Scheme 1 herein. As shown in Scheme 1, TL is synthesized from tyrosine 1 and linoleic acid by first protecting the tyrosine carboxylic group by formation of the tyrosine methyl ester 2, followed by coupling of the protected tyrosine with linoleic acid, forming the amide 3, and finally, hydrolyzing the methyl ester to yield TL 4. TCL is synthesized from TL by first protecting the tyrosine hydroxy group in the aromatic ring to form the silylated derivative 5, followed by reaction with cholesterol to obtain the silylated derivative 6, and removal of the protecting silyl group from 6 to produce TCL 7. TGL is prepared in the same way as TCL by reacting the silylated derivative 5 with 2′-deoxyguanosine and removal of the silyl group from the product thus formed.

In designing the markers of the present invention, the objective was to synthesize and test markers for oxidative stress that would enable the measurement of stress levels, identification of the type of ROS/RNS involved, and characterization of the damaged products and their formation kinetics.

For this purpose, we selected the amino acid tyrosine (Ty), the PUFA linoleic acid (LA) and cholesterol, that are sensitive to ROS/RNS and have been extensively investigated for their reaction products with oxidants (Aviram and Fuhrman, 1998; Chapman et al., 2000; Halliwell, 2000; Jerlich et al., 2000; Szuchman et al., 2003; Botti et al., 2004; Radi, 2004). Furthermore, each one of them represents a major family of the body's building blocks—proteins, unsaturated fatty acids and lipids, respectively, or the DNA when guanosine was inserted instead of cholesterol.

We have recently showed that exposure of tyrosine, linoleic acid and cholesterol, alone or in a mixture, to different types of oxidants results in the formation of different types of oxidative products (Szuchman et al., 2003). In the present invention, we changed the concept by designing an exogenous marker for oxidative stress constructed from tyrosine and linoleic acid and, optionally, cholesterol or 2-deoxyguanosine (connected by covalent bonds), each component representing an essential biomolecule of body-building. Such a designed marker can be used to characterize pathological conditions associated with oxidative stress, based on the type of oxidized products formed.

The markers were designed by connecting two (Ty and LA) or three (Ty, LA and either cholesterol or 2′-deoxyguanosine) well-known endogenous molecules together into one new exogenous molecule. The idea was that any alteration which may occur to the marker(s) (oxidation) as a result of incubating it with cells, organs or other biological sample (such as blood) could be attributed to the ROS/RNS present in the biological sample at the time where the experiment is performed. In addition, since most biological samples contain linoleic acid, tyrosine and cholesterol or guanosine, by using the marker, these endogenous compounds already present in the sample can be easily distinguished from the markers and their oxidized products.

After combining them into one molecule (TL, TCL or TGL), the active sites of tyrosine, i.e. the aromatic ring, and of linoleic acid, i.e. the two double bonds, remained intact. Cholesterol active sites are mostly the double bond at position 5 and the allylic position 7. In addition, the strong amide bond connecting the tyrosine and the linoleic acid is not likely to be easily hydrolyzed chemically or enzymatically by proteases, and the ester bond, connecting the cholesterol or the 2′-deoxyguanosine to the carboxyl group of the tyrosine, is apparently sufficiently strong not to hydrolyze during the incubation time (2-4 h) with the biological sample.

The molecules composing the designed markers, namely Ty, LA and, optionally, cholesterol or 2′-deoxyguanosine, are known to be easily oxidized under oxidative stress and to generate specific oxidized products, depending on the type of ROS/RNS present in vivo. The use of TL constructed from Ty and LA, TCL constructed from Ty, LA and cholesterol, or TGL constructed from Ty, LA and 2′-deoxyguanosine enables us to distinguish them and their oxidized products from the endogenous Ty, LA, cholesterol and guanosine of the tested sample, and to determine their formation time. Thus, an analysis of the oxidative-stress-dependent fingerprint of the oxidized TL (Ox-TL), oxidized TCL (Ox-TCL) or oxidized TGL (Ox-TGL) products may enable an in-depth investigation of the oxidative stress process in cells and tissues and may clarify the association between pathological conditions and oxidative fingerprints.

Phagocytes such as monocytes and macrophages are an important source of highly reactive oxidants, generating HOCl from hydrogen peroxide (H₂O₂) and chloride, and peroxynitrite (ONOO⁻) from nitric oxide and superoxide (O₂⁻.). Exposure of low-density lipids (LDL) to HOCl results in the chlorination and oxidation of their protein and lipid constituents, causing LDL aggregation, and increased cellular uptake by macrophages (Hazen and Heinecke, 1997; Hazell et al., 1994). In a recent study, exposure of LDL to human monocytes resulted in LDL lipid peroxidation and protein nitration. Moreover, incubation of LDL with myeloperoxidase (MPO), known to generate RNS, resulted in increased LDL uptake by macrophages and formation of foam cell (Heller et al., 2000), the hallmark of early atherosclerosis development.

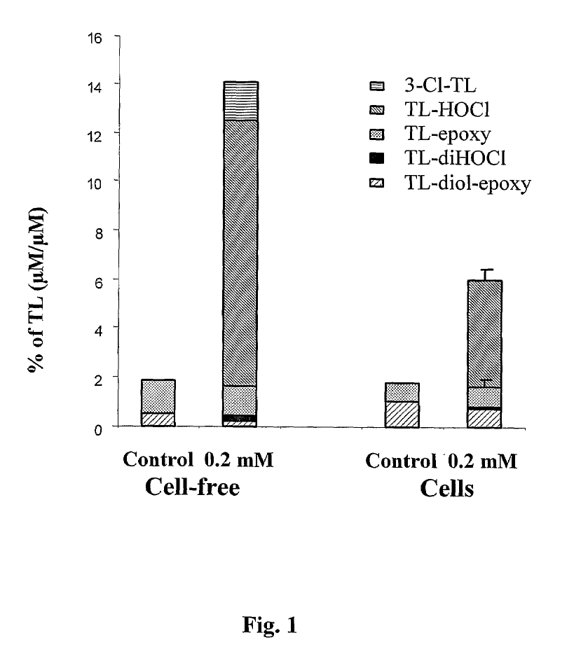

According to the present invention, the effect of HOCl-induced TL oxidation differed significantly when the TL was incubated with macrophages relative to direct treatment of TL with HOCl. The oxidation of TL was significantly retarded in the cell system, by 57%, and unlike the cell-free system, no 3-Cl-TL was detected (FIG. 1). These results could be explained by the cells' buffering effect on the extent of TL oxidation in general, due to either its reductant content (low- and high-molecular-weight antioxidants) or the presence of other substances which could compete with TL for the HOCl, thereby reducing its availability to the former. Another important finding was the selectivity of the reactions in the cells: under our experimental conditions, only the unsaturated fatty acid reacted, but not the tyrosine (no 3-chloro-tyrosine formed) and, thus, under the experimental conditions, in cells, linoleic acid was the favored molecule for reactions with HOCl.

Copper (II) ions are widely used to initiate lipid peroxidation in model systems, particularly in studies of LDL oxidation (Lynch and Frei, 1995; Aviram et al., 1996; Abuja et al., 1997; Burkitt, 2001; Fuhrman et al., 2004). Copper (II) ions can induce lipid peroxidation in the absence of an additional reducing agent, and it has often been assumed that this involves the reduction of copper (II) by a pre-formed lipid hydroperoxide, resulting in the generation of a peroxyl radical (ROO.), which may then initiate further cycles of peroxidation (Cadenas and Sues, 1998).

Incubation of TL with cells followed by their oxidation with copper ions, and a comparison of the results to similar experiments in the cell-free system, revealed that cells buffer the effects of oxidants such as HOCl and copper ions. The present results show that TL-epoxide is formed in relatively large amounts in the cell-free system, but its formation is not associated with the presence of oxidant (1.8% in the control and 2.3% in the presence of copper ions), whereas in cells the amount of epoxidation diminishes considerably (0.6%) and again, this amount is not related to the presence or absence of copper ions. Exposing linoleic acid to air is sufficient to oxidize part of it to linoleyl hydroperoxide (L-OOH), a reaction which is accelerated in the presence of copper ions. The present study shows that TL in cells does not oxidize to TL-OOH despite exposure of the cells to air for 10 h, and no TL-OOH was detected in the controls (no oxidant), whereas the addition of copper ions resulted in TL-OOH formation in the cell system. These experiments indicate that, in addition to the buffering effect of the cells, the copper ions, under our experimental conditions, oxidize linoleic acid but do not affect tyrosine.

Several RNS derived from NO (such as ONOO—) are strong oxidants, which can oxidize lipids, proteins and DNA and are generated in vivo under pathological conditions such as inflammation (Eiserich et al., 1998) and atherosclerosis (Beckmann et al., 1994). SIN-1 at physiological pH decomposes to form nitric oxide and O₂⁻. (Moore et al., 1995), and both can react with each other to form a powerful oxidant, ONOO⁻. In the present invention, using the exogenous marker TL, the effect of SIN-1 in cells was tested and the results compared with its effects in a cell-free system. Similar to the two previous inducers tested, HOCl and copper ions, the amount of SIN-1-induced TL oxidation in cells was significantly lower (3.9 to 1.9 times) than the Ox-TL formed in the cell-free system. These observations were in agreement with our expectations that, in cells, the level of Ox-TL would diminish relative to the cell-free system due to the presence of many other potential reactive compounds, such as antioxidants. Furthermore, SIN-1 decomposes to O₂⁻., which can be removed by the cell's SOD (superoxide dismutase), thus decreasing the amount of ONOO⁻ formation.

Another important finding of the present invention is that using the TL marker, one may be able to identify the type of oxidant (ROS/RNS) affecting the cells at any given time, based on the type of Ox-TL products formed after incubating TL with the cells. Thus, exposing cells to HOCl or copper ions resulted in exclusive attack on the linoleic acid residue of the TL. With HOCl, an addition to the linoleic acid double bond (TL-HOCl) was preferred, and with copper ions, the formation of a linolyl hydroperoxide residue (TL-OOH) was favored. In contrast, when an RNS (SIN-1) was the inducer, the tyrosine moiety of TL was most reactive with predominantly a nitration reaction occurring on the tyrosine aromatic ring and NO₂-TL formation.

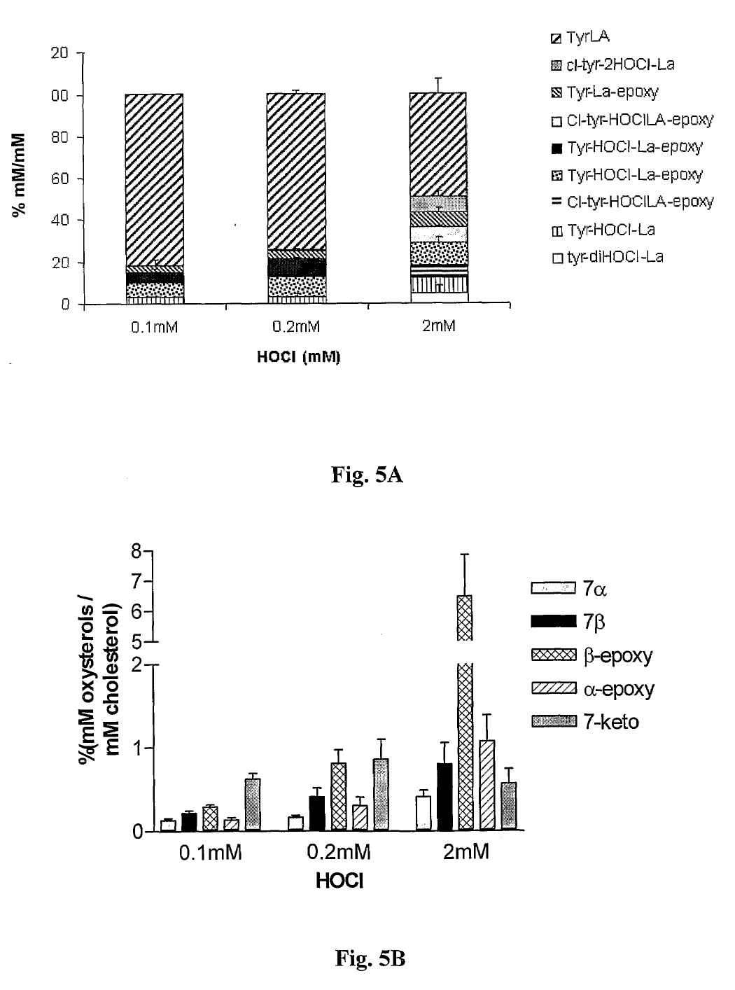

Mouse peritoneal macrophages (MPM) from atherosclerotic E⁰ mice are known to produce ROS/RNS at an increased rate relative to BALB/c control mice, as evidenced by enhanced plasma lipoprotein oxidation (Hayek et al., 1994), and by the accumulation of oxysterols and lipid peroxides in their macrophages (Maor et al., 2000; Vaya et al., 2000; Rosenblat and Aviram; 2002). MPM from atherosclerotic or control mice were incubated with TL to determine whether the cells can oxidize the TL with no addition of any exogenous oxidants, and if the TL marker can distinguish between two types of cells which differ in their levels of oxidative stress. Our results indicated that the TL is sufficiently sensitive to relay this information. TL was oxidized by both cell types, but MPM from E⁰ were shown to produce considerably more TL-OOH than the control cells (MPM from BALB/c) (1.8% vs. 0.2%, FIG. 4), and the type of oxidative products formed, at least initially, clearly originated from the peroxidation reactions. The amount of TL-epoxide detected was less significant; in agreement with our finding that linoleic acid can be oxidized to epoxide, even in a solution that is open to the air. It is interesting to note that when macrophages from E⁰ mice at 4 months of age were tested for their Ox-TL content (older E⁰ mice are expected to be more atherosclerotic), the differences in the amount of TL-OOH between BALB/c and E⁰ mice were even more significant: TL-OOH could not be seen in the MPM in control BALB/c mice, whereas 1.94% TL-OOH was observed in MPM from E⁰ mice. In both mice MPM, we were also able to detect TL-HOCl in about the same amounts (0.1% μM).

It may be concluded that while epoxidation of linoleic acid (formation of TL-epoxide) is a process not necessarily related to in vivo oxidative events, reaction of TL with HOCl to yield TL-HOCl may be a sign of inflammation-induced oxidative stress, and detection of TL-OOH above a certain amount may be a sign of oxidation-induced atherosclerosis.

The analysis of Ox-TCL is performed by first hydrolyzing the ester bond connecting the cholesterol residue to the carboxyl group of the tyrosine, and afterwards detecting the Ox-TL and oxysterols separately, after cholesterol silylation with BSA for 40 min in 80° C., using LC-MS-MS and GC-MS techniques, respectively.

Incubation of TCL with blood samples from hypercholesterolemic patients demonstrates that not much oxidation takes place in hypercholesterolemia. In particular, although blood cells of hypocholesterolemic patients oxidize TL forming hydroperoxide (TL-OOH) and epoxide (TL-epoxy) significantly more than the cells of healthy subjects, combining the total amounts of Ox-TL or oxysterols in serum and cells (total in blood) reveals less pronounced significance between the two groups. Similarly, a minor significant difference was observed in the total amount of oxysterols between the two groups

Analysis of the oxidation products of the TL moiety of the TCL after its incubation with blood samples from diabetic patients revealed considerable higher amounts of TL-OOH, TL-epoxy and an additional adduct of the linoleic acid (formed by simultaneous epoxidation, hydroperoxidation and addition of HOCl) compared to healthy individuals, while oxidation of the cholesterol moiety resulted in high amount of oxysterols (mainly 7β-, β and α-epoxy-, and mostly 7-keto-cholesterol). However, it can be noted that the differences in total Ox-TL and oxysterols vs. total TL and cholesterol, respectively, in diabetic patients vs. healthy individuals, was much more significant in the blood cells than in the serum. In this connection, it is worth noting that after incubation of the TCL with blood samples from diabetic patients, which received for one week pomegranate juice supplementation (known to improve lipid profiles in diabetic patients with hyperlipidemia), remarkably lower amounts of TL-OOH and TL-epoxy were found, while less significant decrease in oxysterols (mainly 7β-, α-epoxy- and 7-keto-cholesterol) was detected in the blood cells.

Incubation of TCL with blood samples from Parkinson patients was found to induce an increased formation of TL-epoxy as well as oxysterols. However, TL-OOH, which is a major product in hypercholesterolemic and in diabetic patients, was not formed at all and, unlike in hypercholesterolemic and in diabetic patients, the major oxysterols formed were 7α- and 7β-hydroxy-cholesterol and not the 7-keto-cholesterol.

The analysis of Ox-TGL is performed as the analysis of Ox-TCL by first hydrolyzing the ester bond connecting the 2′-deoxyguanosine residue to the carboxyl group of the tyrosine, and afterwards detecting the Ox-TL and Ox-guanosine separately. However, the analysis may also be performed directly, without the hydrolyzing step. The major Ox-guanosine formed is the 8-hydroxy- or the oxo-guanosine.

Incubation of TGL with saliva samples from periodontal cancer patients was found to induce significantly increased formation of 8-oxo-guanosine, while no significant increase was detected in the levels of Ox-TL in comparison to saliva sample from healthy individuals.

The method of the present invention is useful in the diagnosis or for monitoring efficacy of treatment of diseases, disorders and pathological conditions associated with oxidative/nitrosative stress such as, but not limited to, cardiovascular diseases such as atherosclerosis, ischemia/reperfusion injury, restenosis and hypertension; cancer; inflammatory diseases such as acute respiratory distress syndrome (ARDS), asthma, inflammatory bowel disease (IBD), dermal and ocular inflammation and arthritis; metabolic diseases such as diabetes; and diseases of the central nervous system (CNS) such as amyotrophic lateral sclerosis (ALS), Alzheimer's disease, Parkinson's disease, and stroke.

As illustrated in Table 8, following IV injection to mice, the markers of the invention are distributed almost all over the body, however the accumulation level in each one of the internal organs is different and time-dependent. Thus, in order to identify pathological conditions using the markers of the present invention, the biological sample may be a biopsy from almost any organ of the patient, i.e. from the liver, kidney, spleen, lungs, skeletal muscle, ovary, intestine and pancreas. However, in preferred embodiments of the present invention, the biological sample is blood, urine or saliva sample.

The markers of the invention may also enable to define specific oxidative stress fingerprints with respect to specific oxidative stress-related pathological conditions, namely, the specific oxidized products profile obtained from each one of said markers and associated with a specific pathological condition. Thus, by defining their specific oxidation products fingerprints, said markers may also enable the identification of a specific disease, disorder or pathological condition associated with oxidative/nitrosative stress.

The invention will now be illustrated by the following non-limiting Examples.

### EXAMPLES

In the examples, the compounds of formula I, the starting compounds and the intermediates are identified by the Arabic numbers 1-8 in bold. The full chemical structures are depicted in Schemes 1-2.

### Materials and Methods

### (i) Materials.

Linoleic acid, N-t-BOC-L-tyrosine (tyrosine-BOC), 3-chlorotyrosine, 3-nitrotyrosine, butylated hydroxyanisole (BHA), BHT, BSA, EDTA, sodium hypochlorite (NaOCl), ph₃P, NBS, pyridine and XTT were purchased from Sigma Chemical Co. (St. Louis, Mo.). SIN-1 was obtained from Acros Organics. PBS, DMEM, RPMI-1640 medium, HBSS, FCS (heat-inactivated at 56° C. for 30 min), penicillin, streptomycin, nystatin, L-glutamine, and sodium pyruvate were obtained from Biological Industries (Beit Haemek, Israel). J-774 A.1 murine macrophage-like cell line was purchased from the ATCC (number TIB-67).

### (ii) TL Distribution and Cytotoxicity in Cells.

A J-774 A.1 murine macrophage-like cell line was cultured at a concentration of 3×10⁶ cells/ml in DMEM containing 5% FCS, 100,000 U/l penicillin, 100 mg/l streptomycin, and 2 mM glutamine. The cell suspension was dispensed into a six-well plate and pre-incubated in a humidified incubator (5% CO₂, 95% air) at 37° C. for 24 h. After 1 day, the medium was changed to HBSS and the cells were supplemented with 40 μM TL dissolved in DMSO and incubated at 37° C. After 3 h, the medium was collected and the cells were washed with 0.5 ml HBSS. Membrane and cytosol fractions were separated as described elsewhere (Nunoi et al., 1988). Cells were washed twice with cold PBS and scraped into 10 ml of relaxation buffer (100 mM KCl, 3 mM NaCl, 3.5 mM MgCl₂, 1.25 mM EGTA, 1 mM ATP, 10 mM Hepes, pH 7.4) containing 1 mM PMSF and 100 μM leupeptin at 4° C. Sonication for 20 s on ice yielded about 95% cell breakage. Nuclei, granules, and unbroken cells were removed by centrifugation (2 min, 15,600 g, 4° C.). The supernatant was centrifuged in a Beckman Airfuge (30 min, 134,000 g) to obtain a cell-membrane pellet and a cytosolic supernatant. Membranes and cytosolic fractions were extracted in a hexane:isopropanol solution (3:2, v/v) followed by acidification with H₂SO₄ in DDW (1:2000) to pH 3. TL concentration was determined by LC/MS/MS.

Cytotoxicity was measured using XTT method, as described: A J-774 A.1 murine macrophage-like cell line was cultured at a concentration of 0.5×10⁶ cells/ml in DMEM containing 5% FCS, 100,000 U/l penicillin, 100 mg/l streptomycin, and 2 mM glutamine. The cells suspension was dispensed into a 96-well plate and pre-incubated in a humidified incubator (5% CO₂, 95% air) at 37° C. for 24 h. After 1 day, the medium was changed to HBSS and the cells were supplemented with 40 μM TL dissolved in DMSO and incubated at 37° C. After 3 h incubation, medium was collected and the cells were washed with 0.2 ml HBSS. Reconstitute XTT was added to the wells in an amount equal to 20% of the culture medium. After two hours incubation at 37° C., the cells viability was determined at 450 nm in Eliza reader.

(iii) Analysis of TL Oxidized Products by LC/MS/MS.

The LC/MS was equipped with HPLC (Waters 2790) with a Waters photodiode array detector (model 996) connected to MS (Micromass Quattro Ultima MS/MS system, UK). The HPLC column was 3.5 μm C18 ODS XTerra (Waters, Mass.) and the eluents were a gradient of solution A (0.1% acetic acid in acetonitrile) and solution B (0.1% acetic acid in DDW) as follows: starting with 40% A; then changing to 60% A for 2 min; and then to 80% A for 10 min. Finally, the column was washed with a solution of 98% A.

MS/MS analysis of the oxidized products was performed in scan mode using electrospray negative ions (ES⁻). The source temperature of the MS was set at 150° C., with a cone gas flow of 75 l/h and a desolvation gas flow of 600 l/h. Peak spectra were monitored between 30 and 600 m/z. Collision-induced dissociation MS was performed, using collision energy of 25-30 eV and 3-3.5 kV capillary voltage. Multiple-reaction-monitoring (MRM) was performed using the same conditions. A calibration curve of TL was run in each analysis.

### (iv) Oxidation of TL in a Cell-Free System.

Oxidation of TL (40 μM) with HOCl (0.1, 0.2 mM), copper ions (CuSO₄, 20 μM) or SIN-1 (0.1 mM) was carried out in HBSS at 37° C. with a vortex. After 30 min, 1 h or 10 h, respectively, the solutions were removed and extracted with hexane:isopropanol (3:2) followed by acidification with a solution of DDW/H₂SO₄ to pH 3-4. The organic layer was then dried (anhydrous sodium sulfate), filtered, evaporated to dryness in an evaporator, and re-suspended in acetonitrile (HPLC-grade) for LC/MS/MS analysis.

For treatment with HOCl, a stock solution of NaOCl was prepared, and the concentration was determined spectrophotometrically (ε₂₉₂=350 M⁻¹cm⁻¹). When copper ions were used as the inducer, a fresh stock solution of CuSO₄ was prepared before each experiment. For treatment with SIN-1, a stock solution of SIN-1 was prepared in 1 N NaOH.

### (v) Oxidation of TL in Cells.

J-774 A.1 murine macrophage-like cells were cultured at a concentration of 3×10⁶ cells/ml in DMEM containing 5% FCS, 100,000 U/L penicillin, 100 mg/l streptomycin, and 2 mmol/l glutamine. The cell suspension was dispensed into a six-well plate and pre-incubated in a humidified incubator (5% CO₂, 95% air) at 37° C. for 24 h. After 1 day, the medium was changed to HBSS. The cells were supplemented with 40 μM TL dissolved in DMSO and incubated at 37° C. Control cells were supplemented with DMSO with no addition of TL. After 3 h, the medium was collected and the cells were washed with 0.5 ml HBSS prior to treatments:

HOCl-Induced Monocytes: HOCl dissolved in HBSS (0.2 mM and 2 mM) were added to PLB-985 pre-monocytes. PLB-985 were incubated and treated as described for J774.A1 cells. After 30 min of incubation, the cells were harvested, extracted, dried and resuspended with acetonitrile for LC/MS analysis.

Copper Ion-Induced Macrophages: 20 μM CuSO₄ dissolved in HBSS was added to J-774 macrophages. After 10 h of incubation, the cells were harvested and extracted twice with hexane:isopropanol (3:2) followed by acidification with H₂SO₄ in DDW to pH 3. The samples were then dried and evaporated, and resuspended in acetonitrile for LC/MS analysis.

SIN-1-Induced Macrophages: SIN-1 dissolved in HBSS (0.1 mM) was added to the J-774 cells. After 30 min of incubation, the cells were harvested, extracted, dried and resuspended with acetonitrile for LC/MS analysis.

Cells containing TL (40 μM) without inducers were used as controls.

### (vi) Mouse Peritoneal Macrophages (MPM).

Apolipoprotein-deficient (E⁰) mice were generously provided by Dr. Jan Breslow from the Rockefeller University (New York, USA). These mice were created by using gene-targeting in mouse embryonic stem cells. They lack apolipoprotein E and are characterized by hypercholesterolemia, oxidative stress and accelerated atherosclerosis (Plump et al., 1992). These mice were compared with BALB/c mice (as controls) of the same age, sex and genetic background.

MPMs were harvested from 2- and 4-month-old BALB/c and E⁰ mice 3 days after intraperitoneal injection of 3 ml 4% thioglycolate. The cells were washed twice with PBS, resuspended at a concentration of 3×10⁶ cells/ml in DMEM containing 10% FCS, 100,000 U/l penicillin, 100 mg/l streptomycin and 2 mM glutamine. The cell suspension was dispensed into a six-well plate and incubated in a humidified incubator (5% CO₂, 95% air) at 37° C. for 4 h. The dishes were washed once with 5 ml DMEM to remove non-adherent cells. The monolayer was then incubated with 40 μM or 80 μM TL under similar conditions. After 20 h, the medium was removed and the cells were harvested and extracted twice with hexane:isopropanol (3:2) followed by acidification with H₂SO₄ in DDW to pH 3. The samples were then dried and evaporated, and resuspended in acetonitrile for LC/MS analysis.

(vii) HPLC and LC-MS Measurements.

In the HPLC measurements, the products were identified by using a DAD-HPLC (HP-1100) with C-8 reverse phase column 5 μm (125 mm length; 4 mm diameter). The mobile phase was a mixture of acetonitrile and water with a flow of 1 ml/min and a gradient:

| | | | |
|---|---|---|---|
| 0 | min | 20% acetonitrile | 80% water |
| 10 | min | 80% acetonitrile | 20% water |
| 20 | min | 97% acetonitrile | 3% water |

The LC-MS measurements were done using a DAD-HPLC-MS (water 2790 HPLC with Waters photodiode array detector 996 and Micromass Quattro Ultima MS). The separation column and the mobile phase gradients were the same as in the HPLC measurements.

(viii) Synthesis of 3-Nitro- and 3-Chloro-Tyrosine N-Linoleyl for use as Standards.

N-Linoleyl 3-nitro-tyrosine (3-NO₂-TL) and N-linoleyl 3-chloro-tyrosine (3-Cl-TL) were synthesized using the same method as for the synthesis of TL, which is described below (see Example 1). Both compounds were used as standards.

### (ix) Statistical Analysis.

The relative amount of the oxidation products was calculated as μM percentage of the unoxidized TL in the sample. Each experiment was repeated at least three times and Student t-test was used to analyze the significance of the results, which are given as mean±SD.

### Example 1

### Synthesis of N-Linoleyl Tyrosine (TL), 4

The synthesis of the title compound was carried out in three steps as depicted in Scheme 1 hereinafter.

1(i) Synthesis of Tyrosine Methyl Ester, 2.

Concentrated (96%) H₂SO₄ (2 ml) was added dropwise to a heterogenic solution of tyrosine 1 (2 g, 11 mmol) in methanol (30 ml). The mixture was refluxed for 4 h, and the solution was concentrated by evaporating the methanol. The solution was brought to pH of about 10 half by a saturated solution of sodium carbonate and extracted with three portions of DCM. The organic phase was evaporated to a white solid (1.5 g, 7.68 mmol, 70% yield), which was identified by FTIR, UV, LC/MS and NMR as tyrosine methyl ester 2.

1(ii) Synthesis of N-Linoleyl Tyrosine Methyl Ester, 3 (Froyen, 1997).

NBS (1.25 g, 7 mmol) was added in one portion to a stirred solution of triphenylphosphine (ph₃P) (1.71 g, 6.5 mmol) and linoleic acid (1.71 g, 6.10 mmol) in dried DCM (5 ml) at 0° C. The solution mixture was set aside at room temperature, while a new solution was prepared: tyrosine methyl ester 2 (1.14 g, 5.9 mmol) and pyridine were dissolved in dried DCM. The mixture was cooled to 0° C. and vigorously stirred while the first solution was added dropwise. The solvents were evaporated to give a crude material which was purified by flash chromatography (silica, hexane:acetone 70:30, v/v). Removal of the solvent gave pure TL methyl ester 3 (2.5 g, 5.5 mmol, 94% yield). The product showed a peak at 12.23 min by HPLC and was identified by LC/MS (ES⁻ 456.9) (not shown). ¹H-NMR analysis (CDCl₃, 200 mHz) δₚₚₘ results; 6.89 (d, 2H); 6.67 (d, 2H); 5.87 (d, 2H); 5.31 (m, 4H); 4.8 (q, 1H); 3.71 (s, 3H); 3.00 (m, 2H); 2.74 (t, 3H); 2.14 (t, 2H); 2.00 (m, 4H); 1.59 (m; 4H); 1.25 (s, 14H), 0.82 (d, 2H). IR (neat) νmax 3352, 2925, 1736, 1655, 1517, 1448.

1 (iii) Synthesis of N-Linoleyl Tyrosine (TL), 4.

A solution of 10 ml NaOH (10%) in water was added to TL methyl ester 3 (1 g, 2.18 mmol). The mixture was stirred overnight at room temperature and then the pH was adjusted to 3 with 40% H₃PO₄. The solution was extracted with ethyl acetate (three portions). The organic layer was separated and evaporated to give the product as a colorless oil (0.95 g, 2.14 mmol) at a yield of 99%. The product showed one peak at 10.71 min (HPLC) and was identified by MS (direct injection, ES⁻ 442.6) (not shown). ¹H-NMR analysis (CDCl₃, 200 mHz) δₚₚₘ; 7.17 (d, 2H); 6.65 (d, 2H); 6.22 (d, 1H); 5.31 (m, 4H); 4.8 (q, 1H); 3.02 (d, 2H); 2.73 (t, 2H); 2.16 (t, 2H); 2.03 (m, 6H); 1.23 (s, 14H); 0.85 (t; 3H). IR (net) νmax 3341, 2926, 1723, 1650, 1516, 1445.

### Example 2

### HOCl-Induced Oxidation of TL in a Cell-Free System and in Cell Culture

Treatment of TL (40 μM) in a cell-free system with HOCl (0.2 mM) resulted in the formation of a major product (TL-HOCl) obtained from the addition of one equivalent of HOCl to one of the two linoleic acid double bonds (10.8%, representing 77% of the total Ox-TL detected) (FIG. 1). Another product was 3-chloro-TL (3-Cl-TL), resulting from chlorination on the tyrosine aromatic ring (1.5%). A minor product (0.2%) was obtained as a result of two equivalents of HOCl added to the linoleic acid moiety (TL-diHOCl). An oxidative product containing epoxide on the linoleic acid moiety of the TL was found, with or without added oxidant, in similar amounts (1.3%), suggesting that the epoxide product at the detected level could be formed in all cases under the experimental conditions (FIG. 1). Upon increasing the HOCl concentration to 0.4 mM, a parallel increase in the total amount of Ox-TL was noted, from 14% to 29% (of the total TL added), with the major Ox-TL products being TL-HOCl (up to 20%), 3-Cl-TL (up to 4.3%) and TL-diHOCl (up to 2%) (data not shown).

We next studied the oxidation of TL in a cell system of J-774 A.1 macrophages. Incubation of TL (40 μM) with the cells, followed by cell oxidation with HOCl (0.2 mM) for 30 min and then extraction of the oxidized materials and their analysis by LC/MS/MS showed a significantly decreased level (2.3-fold) of Ox-TL products in the cells relative to their amount in the cell-free experiments. In addition, practically only TL-HOCl was formed. The other two minor Ox-TL products were also present in the absence of oxidant, i.e., TL-epoxy and TL-epoxy-diol, in which the second double bond of the linoleic acid is converted to diol. Thus, their formation was not necessarily linked to the addition of HOCl.

The Ox-TL products were identified by either synthesizing them (3-Cl-TL, 3,5-dichloro-TL), or deducing them from their MS spectra in a scan mode using electrospray negative ions (ES⁻), as well as from fragmentation of the molecular ions, using daughter ion and/or multiple-reaction-monitoring (MRM) methods. Thus the LC/MS/MS analysis of HOCl-induced oxidation of TL in cells revealed four compounds: (i) Tyrosine-dihydroxy-epoxy-N-linoleyl (TL-di-OH-epoxy) with a molecular ion of m/z 492 (M⁻¹) and a major fragmentation of m/z 180, corresponding to tyrosine (M⁻¹-linoleic acid); (ii) 3-chloro-TL (3-Cl-TL), in which chlorination of the tyrosine aromatic ring occurs, with a molecular mass of m/z 476 (M⁻¹) and major fragmentations at m/z 432 (M⁻¹-CO₂) and m/z 214 (corresponding to chloro-tyrosine); (iii) Tyrosine-HOCl-N-linoleyl (TL-HOCl), from the addition of HOCl to the linoleic acid moiety, with a molecular mass of m/z 494 (M⁻¹) and major fragmentations at m/z 458 (M⁻¹-HCl), m/z 397 (fragmentation between C₁₁-C₁₂) and m/z 180; and (iv) TL-di-HOCl with a molecular mass of m/z 546 (M⁻¹) and fragmentation ions at m/z 510 (M⁻¹-HCl), m/z 474 (M⁻¹-2*HCl) and m/z 180.

### Example 3

### The Oxidative Effect of Copper Ions on TL in a Cell-Free System and in Cell Culture

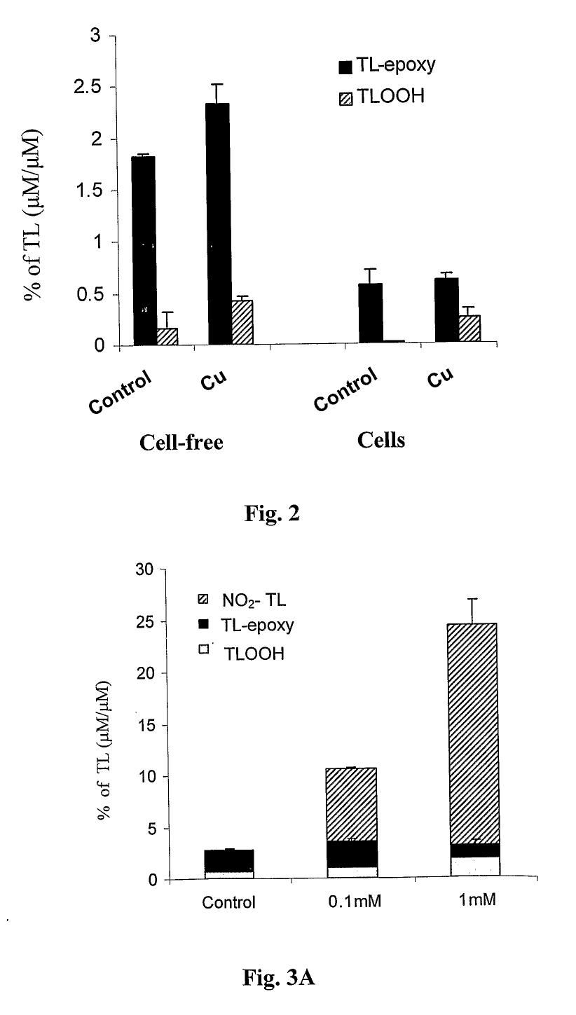

Upon exposure of TL to copper ions (20 μM) during 10 h in a cell-free system, only two major Ox-TL products were obtained: TL-epoxy (2.3%) and TL-hydroperoxide (TL-OOH, 0.42%). Both compounds were also present in the control, where no copper ions had been added (FIG. 2), indicating that these types of oxidation can occur even when samples of TL are in a solution that is open to the air. In similar experiment in cells (J-774 A.1 macrophages), the amount of TL-epoxy was about three-fold less than its amount in the cell-free system. Addition of copper ions to the cells did not affect the amount of TL-epoxy formed. The level of TL-OOH detected in the cells was also less than that seen in the cell-free experiment (0.01% and 0.15%, respectively), but it increased significantly upon addition of copper ions to the cells (from 0.01% to 0.24%). The LC/MS/MS analysis of copper ions-induced oxidation of TL in cells revealed two compounds: (i) TL-epoxy with a molecular ion of m/z 458 (M⁻¹) and major fragmentations at m/z 372 (M⁻¹-C₆H₁₄) and m/z 180 (tyrosine); and (ii) TL-hydroperoxide (TL-OOH) with a molecular ion of m/z 474 (M⁻¹) and major fragmentations at m/z 388 (M⁻¹-C₆H₁₄) and m/z 180 (tyrosine), showing that the tyrosine aromatic ring remained unaffected.

### Example 4

### SIN-1-Induced Oxidation of TL in a Cell-Free System and in Cell Culture

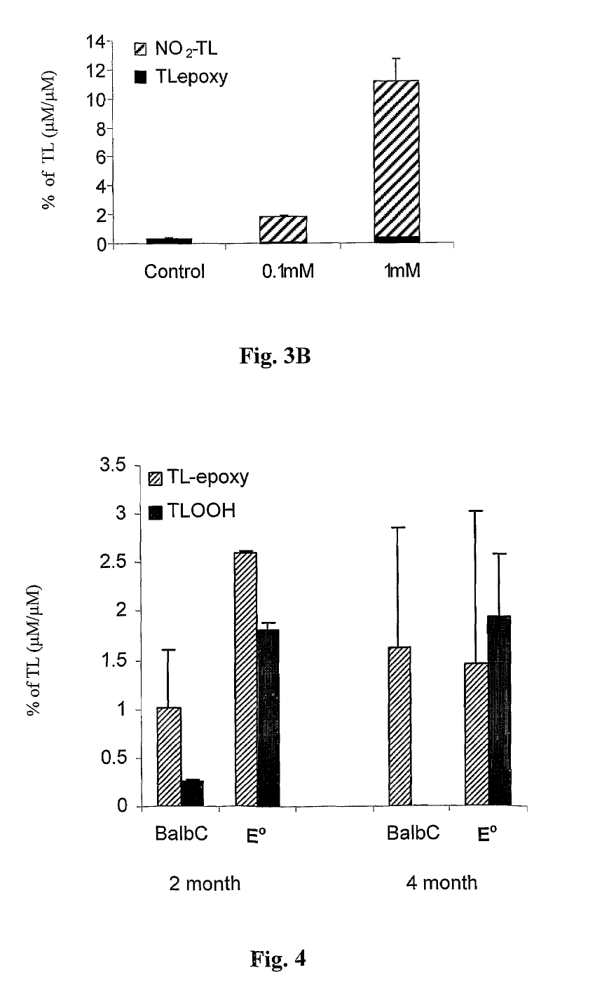

Treatment of TL (40 μM) in the cell-free system with 0.1 or 1.0 mM SIN-1 yielded one major Ox-TL product (FIG. 3), from mono-nitration of the tyrosine aromatic ring (NO₂-TL, 7.0% and 21.2% Ox-TL/total TL, obtained with 0.1 and 1.0 mM SIN-1, respectively). In addition, a very minor Ox-TL product was formed where the linoleic acid moiety was oxidized to hydroperoxide (TL-OOH), with the OOH connected to carbon 13 (1.8% TL-OOH/total TL, using 1 mM SIN-1). As shown with the other oxidants, epoxidation of linoleic acid took place independent of the presence or type of oxidant used. Addition of SIN-1 to J-774 A.1 cells, as previously seen for HOCl and copper ions, resulted in a significant decrease in TL oxidation, by 3.9- and 1.9-fold using 0.1 and 1.0 mM SIN-1, respectively. In parallel, the amount of epoxide detected in cells diminished significantly (from 2.1% to 0.4% in the cell-free system and control cells without oxidant, respectively) and no TL-OOH could be found. Ox-TL products were identified using the same analytical techniques as with the other inducers, i.e. by comparison with standard (NO₂-TL) and from LC/MS/MS analysis. These products were (i) TL-epoxy (M⁻¹=458) and its fragmentation ion at m/z 180 (tyrosine); (ii) nitro-TL (NO₂-TL) with nitro groups on the aromatic ring, a molecular mass of m/z 487 (M⁻¹) and major fragmentations at m/z 443 (M⁻¹-CO₂) and m/z 225 (nitro-tyrosine); and (iii) TL-hydroperoxide (TL-OOH) with a molecular mass of m/z 474 (M⁻¹) and an —OOH group on carbon 13, as evidenced by its fragmentation between C₁₁-C₁₂ at m/z 346 (M⁻¹-C₇H₁₄O₂) and the tyrosine peak (m/z 180).

### Example 5

### The Effect of Mouse Peritoneal Macrophages (MPM) on TL Oxidation

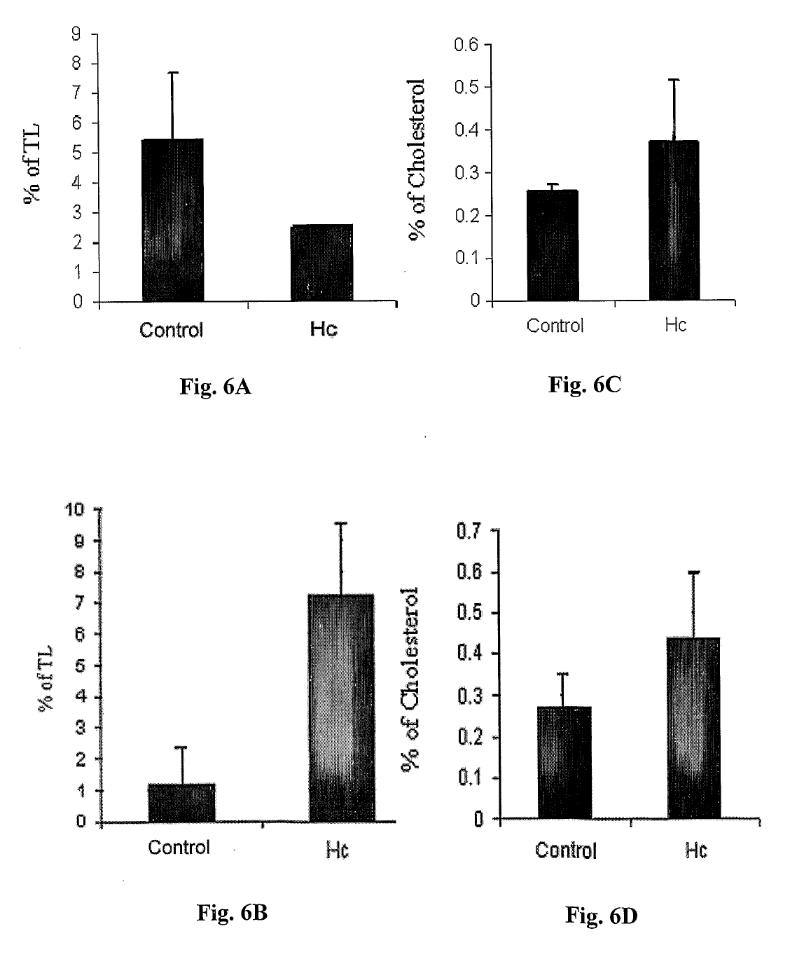

MPM harvested from BALB/c and E⁰ mice (at both 2 months and 4 months of age) after induction of inflammatory conditions (thioglycolate injection) were used to test whether activated macrophages can induce oxidation of the TL marker. FIG. 4 demonstrates that, in both types of mice, two Ox-TL products were detected: TL-epoxy and TL-hydroperoxide (TL-OOH). Both Ox-TL products were present at significantly higher levels in E⁰ MPM vs. control BALB/c MPM (4.4% vs. 1.2%, sum of Ox-TL/total TL). The predominant product was TL-OOH (1.8% in E⁰ vs. 0.2% in the control, BALB/c MPM) (FIG. 5). These results clearly indicate that the TL marker is sensitive enough to identify increased oxidative stress stimulated in macrophages from atherosclerotic mice.

### Example 6

### Synthesis of N-Linoleyl Tyrosine Cholesteryl Ester (TCL), 7

The synthesis of the title compound was performed as depicted in Scheme 1 hereinafter, starting from compound 4.

6(i) Synthesis of N-Linoleyl 4-t-Butyldimethylsilyloxy-Tyrosine, 5.

A solution of imidazole (140 mg, 2.1 mmol) in dry DMF (1 ml) was added dropwise to a solution of TL 4 (90 mg, 0.2 mmol) in dry DMF (2ml) at 0° C. The mixture was stirred for 10 minutes and a solution of t-butyldimethylsylilchloride (TbdmsCl) (220 mg, 1.5 mmol) was added dropwise. The mixture was left to reach the room temperature and stirred over night under nitrogen atmosphere. A solution of K₂CO₃ (10% in water) was added to the mixture and stirred for 30 minutes. The pH of the solution was adjusted to ˜3 with a solution of 40% H₃PO₄ and the solution was extracted by three portions of diethyl ether. The solvents were removed and the product purified by flash chromatography (silica, methanol:dichloromethane 10:90). Removal of the solvent gave the title product 5 (105 mg, 0.19 mmol, 94% yield). The product showed a peak at 16.90 min (HPLC), and was identified by MS (direct injection, ES⁻ 557), IR (net) νmax 3390, 2956, 1716, 1651, 1510, 1455.

6(ii) Synthesis of N-Linoleyl 4-t-Butyldimethylsilyloxy-Tyrosine Cholesteryl Ester, 6.

A solution of EDC (103 mg, 0.5 mmol) was added dropwise to a stirred solution of N-linoleyl 4-t-butyldimethylsilyloxy-tyrosine 5 (60 mg, 0.11 mmol), cholesterol (125 mg, 0.3 mmol) and PTSA (41 mg, 0.2 mmol) in DCM (10 ml). The mixture was refluxed over night under nitrogen atmosphere. Another portion of EDC and PTSA was added and the mixture was refluxed over night. Removal of the solvent gave the title product 6 (40 mg, 0.041 mmol, 40% yield), that was purified by flash chromatography (silica, hexane:ethyl acetate 80:20). The IR (net) of the product is νmax 3326, 2931, 1732, 1651, 1509, 1463, 1259.

6(iii) Synthesis of N-Linoleyl Tyrosine Cholesteryl Ester (TCL), 7.

1 ml of tetrabutylammonium fluoride (TBAF) solution (1M in tetrahydrofuran, THF) was added to a solution of compound 6 (40 mg, 0.041 mmol) in 10 ml of THF at 0° C. The mixture was stirred at 0° C. for 15 minutes. The solution was washed with two portions of water. The solvents were removed and the product was purified by flash chromatography (silica, hexane:ethyl acetate 80:20). Removal of the solvent gave the title product 7 (31 mg, 0.038 mmol, 89% yield). The product shows a peak at 27.16 min (HPLC) and was identified by MS (direct injection, ES⁻ 811.5) (not shown). ¹H-NMR analysis (CDCl₃, 400 mHz) δₚₚₘ; 6.94 (d, 2H); 6.70 (d, 2H); 5.90 (d, 1H); 5.83 (s, 1H); 5.32 (m, 4H); 4.80 (q, 1H); 4.62 (t, 1H); 3.04 (m, 2H); 2.75 (t, 2H); 2.29 (d, 2H); 2.15 (t, 2H); 2.02 (m, 6H); 1.83 (s, 3H); 1.57 (m, 12H); 1.21 (m, 19H); 1.07 (m, 7H); 1.00 (s, 6H); 0.85 (m, 12H); 0.66 (s, 3H), IR (net) νmax 3418, 2929, 1721, 1652, 1519, 1464.

### Example 7

### HOCl-Induced Oxidation of TCL in a Cell-Free System

A solution of N-linoleyl tyrosine cholesteryl ester (TCL) (2 mM) in PBS (50 mM pH=6.0) with 0.6 mM Tween-20 was treated with NaOCl, at a final concentration of 0.1, 0.2 and 2 mM at 37° C., while vortex continuously. After 15 min, the TCL was hydrolyzed by adjusting the pH to 13 with 10% NaOH, for 3 hours at 37° C. The solution was then acidified with HCl (0.2 N) to pH=3-4, loaded on Sep-Pak (C-18, Waters) and washed with water following THF. The organic layer was then dried (with sodium sulfate), filtered and evaporated to dryness under nitrogen atmosphere. After hydrolyzing the ester bond of the TCL, the residues were divided for LC-MS-MS analysis of TL and Ox-TL and for GC-MS analysis of oxysterols after cholesterol silylation with BSA for 40 min in 80° C.

### Example 8

### Culture of Monocyte Cells and Introduction of TCL into the Cells

The human myeloid leukemia cell line PLB-985 (pre-monocytes), a generous gift from Dr. Levy R., The Ben-Gurion University (Beer-Sheva, Israel), was cultured in RPMI-1640-glutamine medium supplemented with 10% FCS, 100,000 U/l penicillin, and 100 mg/l streptomycin, at 37° C. in a humidified incubator (5% CO₂, 95% air). Cell cultures were passed twice a week to maintain a cell density between 2×10⁵ and 10⁶/ml.

Introduction of the TCL marker to the cells was done by incubation of the TCL (40 μM in DMSO) into 5×10⁶ cells/well seeded with HBSS in a 24-well plate in humidified incubator (5% CO₂, 95% air) at 37° C. for 4 hours. To find whether the TCL marker is in the membranes or introduced to the cytosol, after the incubation, the media was removed by centrifugation (1500 rpm, 10 min) and the cells were re-suspended with relaxation buffer (100 mM KCl, 4 mM Na Cl, 1.75 mM MgCl₂, 10 mM HEPES, 1.25 mM EGTA, 1 mM PMSF, and 100 M Leupeptin). The cells were then sonicated using W-375 (Ultrasonics Inc.) and the membranes were separated from the cytosols by ultracentrifuge (40000 rpm for 30 min at 4° C.).

### Example 9

### The Effect of Hypercholesterolemic Patients' Blood on TCL Oxidation

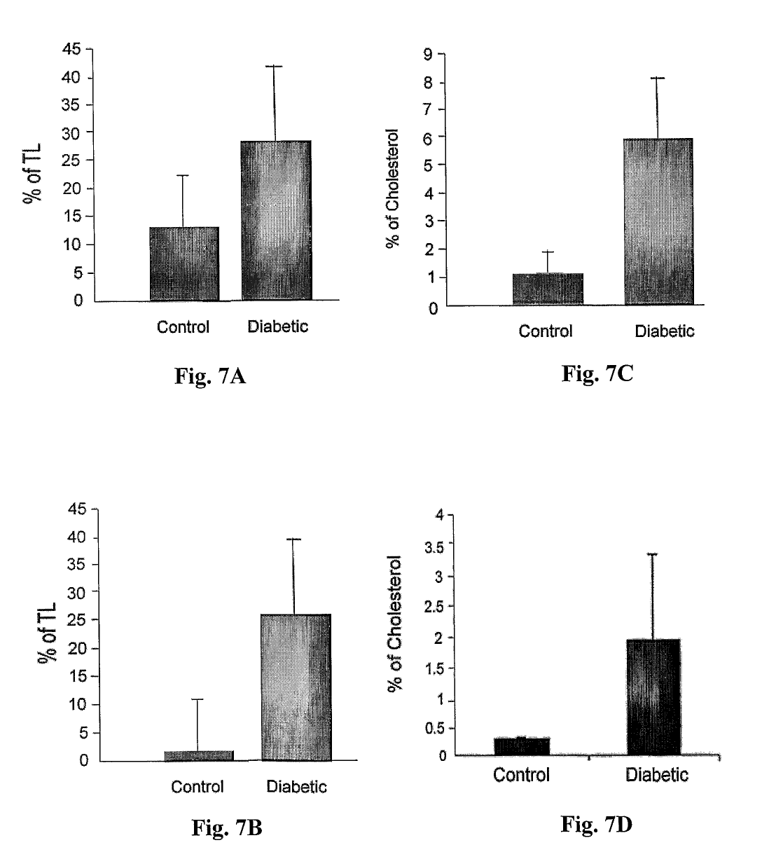

Blood from hypercholesterolemic patients (5) and from healthy subjects (2) were aspirated and immediately added (1 ml) to either tubes containing TCL samples (160 μM) dissolved in DMSO (8 μL), or to tubes containing DMSO (8 μL) as a control. The blood samples were allowed to coagulate during 1 h at room temperature and the plasma was obtained after blood centrifugation. The supernatant and cell debris were collected separately, extracted (chloroform:methanol) and stored at −20° C. and then the cholesterol ester was hydrolyzed from the marker, followed by re-extraction of oxidized TL (ox-TL) and cholesterol (oxysterols). Analysis of the oxidized marker units was performed by injection to LC/MS/MS (for detection of TL and Ox-TL) and to GC/MS (for detection of oxysterols after cholesterol silylation). Analysis of the Ox-TL moiety of the marker reveals that the cells of hypercholesterolemic patients oxidize the TL forming hydroperoxide (TL-OOH) and epoxide (TL-epoxy) significantly more than the cells of healthy subjects. However, combining the total amount of Ox-TL in serum and cells (total in blood) reveals less pronounced significance between the hypercholesterolemic patients and healthy subjects (Table 1 and FIGS. 6A-6B). Similarly, although minor changes were detected in the level of some oxysterols (both in the cells and in the serum), minor significant difference was observed in the total amount of oxysterols between the two groups (Table 2 and FIGS. 6C-6D).

TABLE 1
Ox-TL products vs. TL total amount in hypercholesterolemic
Patients vs. healthy individuals (in μM)

| | TL-OOH | TL-OOH(386) | Tl-epoxy | 527 | sum |
|---|---|---|---|---|---|
| Cells | | | | | |
| Control | 0.387 | 0.067 | 0.510 | 0.325 | 1.289 |
| Hc | 1.310 | 2.062 | 3.865 | 0.104 | 7.341 |
| Serum | | | | | |
| Control | 3.804 | 0.353 | 1.097 | 0.165 | 5.419 |
| Hc | 0.789 | 0.059 | 1.570 | 0.081 | 2.499 |

* The values are the mean of control (2 samples), hypercholesterolemic (5 samples)

TABLE 2
Oxysterols vs. cholesterol total amount in hypercholesterolemic
patients vs. healthy individuals (in μM)

| | 7α | 7β | β-epoxy | α-epoxy | 7-keto | sum |
|---|---|---|---|---|---|---|
| Cells | | | | | | |
| Control | 0.009 | 0.015 | 0.052 | 0.036 | 0.093 | 0.205 |
| Hc | 0.005 | 0.005 | 0.061 | 0.042 | 0.323 | 0.436 |
| Serum | | | | | | |
| Control | 0.017 | 0.014 | 0.109 | 0.030 | 0.086 | 0.256 |
| Hc | 0.003 | 0.005 | 0.072 | 0.070 | 0.217 | 0.367 |

* The values are the mean of control (2 samples), hypercholesterolemic (5 samples)

### Example 10

### The Effect of Diabetic Patients' Blood on TCL Oxidation

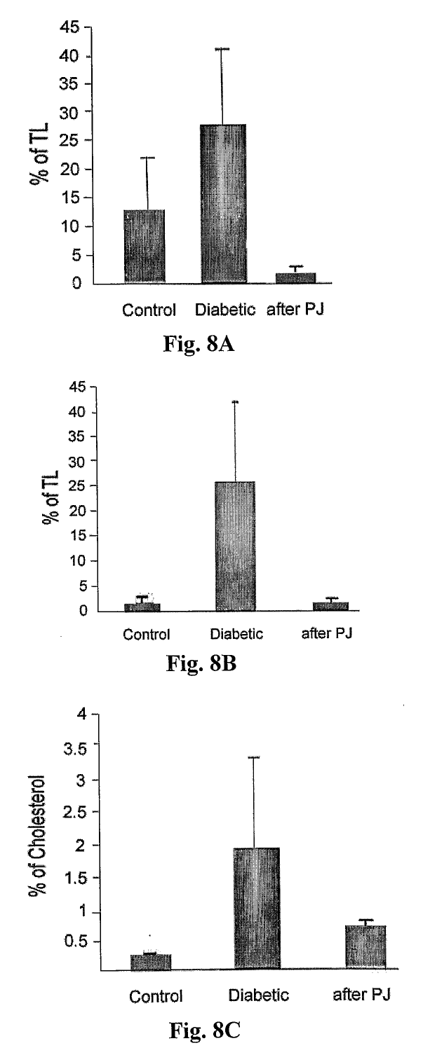

TCL was incubated with blood from diabetics (3) and from healthy subjects (2) as in Example 9 above for hypercholesterolemic patients. Analysis of the Ox-TL moiety of the marker in cells and serum reveals a considerable higher amount of hydroperoxide (TL-OOH), epoxide (TL-epoxy) and an additional adduct of the linoleic acid (formed by simultaneously epoxidation, hydroperoxidation and addition of HOCl) (peak 527) (Table 3). Similarly, high amounts of the oxysterols 7β-, β and α-epoxy-, and mostly 7-keto-cholesterol, were detected in both blood cells and serum (Table 4). However, it can be noted that the differences in total amount of Ox-TL vs. TL and total amount of oxysterols vs. cholesterol in diabetic patients vs. healthy individuals are much more significant in the blood cells than in the serum (FIGS. 7A-7D).

TABLE 3
Ox-TL products vs. TL total amount in diabetic
patients vs. healthy individuals (in μM)

| | TL-OOH | TL-epoxy |
|---|---|---|
| Cells | | |
| Control | 0.527 | 0.700 |
| Diabetic | 1.790 | 23.70 |
| Serum | | |
| Control | 4.158 | 8.385 |
| Diabetic | 18.819 | 9.112 |

* The values are the mean of control (2 samples), Diabetics (3 samples)

TABLE 4
Oxysterols vs. cholesterol total amount in diabetic
patients vs. healthy individuals (in μM)

| | 7α | 7β | 4β | β-epoxy | α-epoxy | 25-OH | 7-keto |
|---|---|---|---|---|---|---|---|
| Cells | | | | | | | |
| Control | 0.012 | 0.033 | 0.015 | 0.011 | 0.143 | 0 | 0.049 |
| Diabetic | 0.007 | 0.234 | 0.001 | 0.140 | 0.284 | 0 | 1.235 |
| Serum | | | | | | | |
| Control | 0.024 | 0.179 | 0.100 | 0.036 | 0.147 | 0.044 | 0.662 |
| Diabetic | 0.079 | 0.271 | 0.096 | 0.399 | 0.447 | 0.023 | 4.635 |

* The values are the mean of control (2 samples), Diabetics (3 samples)

### Example 11

### The Effect of Diabetic Patients' Blood Before and After 1 Week of Pomegranate Juice (PJ) Supplementation on TCL Oxidation

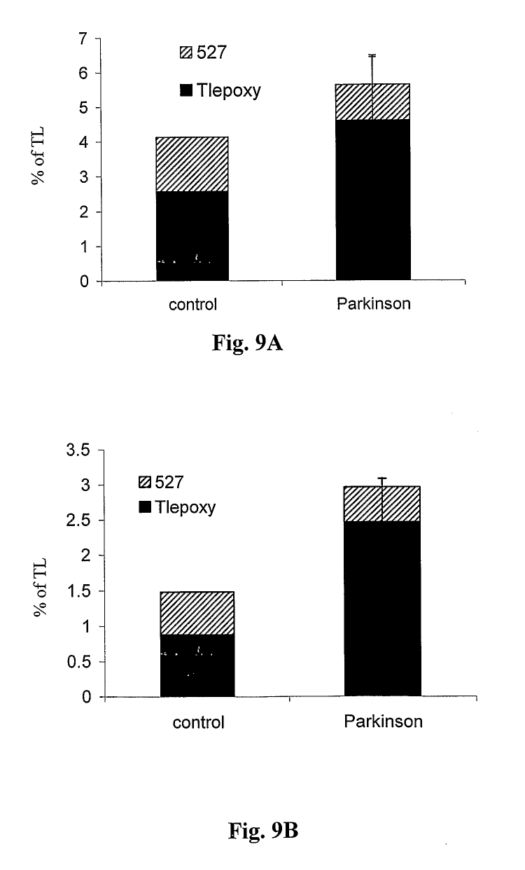

TCL was incubated with blood from diabetic patients before and after 1 week of pomegranate juice (PJ) supplementation (250 ml/day) which is known to improve lipid profiles in diabetic patients with hyperlipidemia, as well as from healthy patients, as in Example 9 above for hypercholesterolemic patients. Analysis of the Ox-TL moiety of the marker in cells and serum shows a remarkable decrease in the amount of hydroperoxide (TL-OOH) and epoxide (TL-epoxy) after 1 week of PJ supplementation (Table 5). Similarly, analysis of the oxysterols in the cells shows that the amount of oxidized cholesterol (7β-, α-epoxy- and 7-keto-cholesterol) also decreased significantly (Table 6), although the differences in levels of total Ox-TL before and after the treatment were much more significant than the differences in the levels of total oxysterols (FIGS. 8A-8C).

TABLE 5
Ox-TL products vs. TL total amount in diabetic patients
before and after 1 week of PJ (in μM)

| | TL-OOH | TL-epoxy |
|---|---|---|
| Cells | | |
| Before PJ | 1.790 | 23.70 |
| After PJ | 0.313 | 0.888 |
| Serum | | |
| Before PJ | 18.819 | 9.112 |
| After PJ | 0.781 | 0.521 |

* The values are the mean of 3 samples

TABLE 6
Oxysterols vs. cholesterol total amount in blood cells of diabetic
patients before and after PJ consumption (in μM)

| | 7α | 7β | 4β | β-epoxy | α-epoxy | 25-OH | 7-keto |
|---|---|---|---|---|---|---|---|
| Before PJ | 0.007 | 0.234 | 0.001 | 0.140 | 0.284 | 0 | 1.235 |
| After PJ | 0.007 | 0.017 | 0 | 0.106 | 0.076 | 0 | 0.477 |

* The values are the mean of 3 samples

### Example 12

### The Effect of Parkinson Patients' Blood on TCL Oxidation

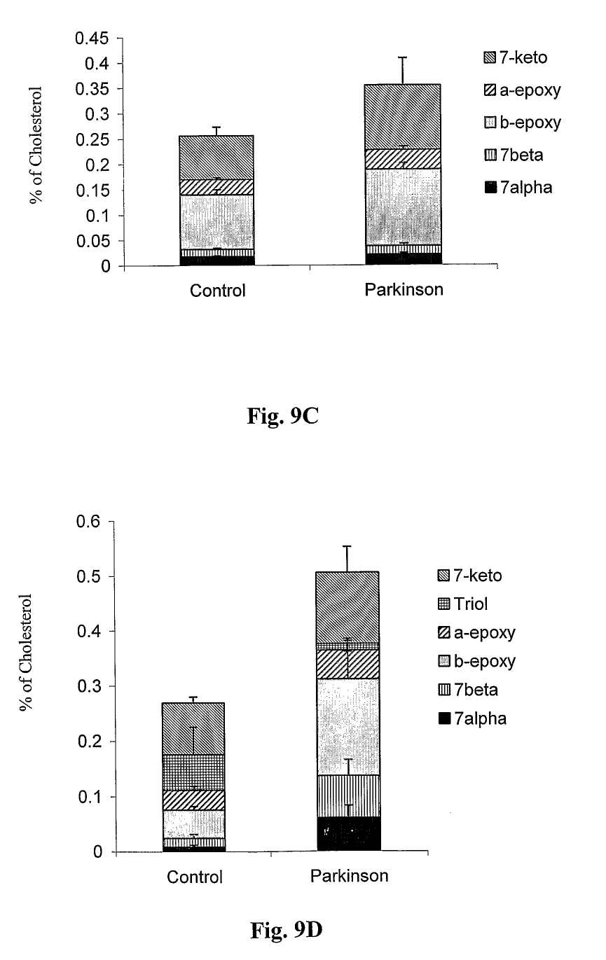

TCL was incubated with blood from Parkinson's patients (3) and from healthy subjects (2) as in Example 10 above for diabetic patients. Analysis of the Ox-TL moiety of the marker in cells and serum reveals relatively moderate increase in total amount of Ox-TL, but it is worth noting that hydroperoxide (TL-OOH), which is very common and major product in hypercholesterolemic and diabetic patients, is not present at all in these samples (FIGS. 9A-9B). Similarly, a moderate increase in total amount of oxysterol was detected although, unlike the situation in hypercholesterolemic and diabetic patients, the major increase was of 7α and 7β hydroxy cholesterol and not of 7-keto-cholesterol (FIGS. 9C-9D).

### Example 13

### Synthesis of N-Linoleyl Tyrosine 2′-Deoxyguanosine Ester (TGL), 8

The starting material for the procedure was N-linoleyl 4-t-butyldimethylsilyloxy tyrosine 5, which was used for the synthesis of the TCL (compound 7).

2′-Deoxyguanosine (100 mg, 0.35 mmol), 1-hydroxybenzotriazole (70 mg, 0.5 mmol) and triethylamine (100 μL, 1.36 mmol) were dissolved in 5 ml DMF under N₂ atmosphere. To the solution were added compound 5 (150 mg, 0.27 mmol dissolved in 2 ml THF) and N-(3-dimethylaminopropyl)-N′-ethylcarbodiimide EDC (300 μl, 2.2 mmol). The resulting solution was stirred at 50° C. for overnight under N₂ atmosphere. TBAF (500 μl of 1M solution in THF) was added to remove the t-butyldimethylsilyl protecting group. The resulting solution was stirred at room temperature for 30 min. NaHCO₃ (20 ml of 5% solution in water) was added and the product was extracted three times with ethyl acetate. The solvents were removed under vacuum and the remaining solid was purified using reverse phase chromatography (silica C18, H2O; ACN) to yield 40 mg product (22% yield). Product 8, in which the 2′-deoxyguanosine is linked through the 6′ OH position, was identified by HPLC (one peak at 9.5 min), Scan LC-MS ES⁻: m/z=169 and Daughter LC-MS ES⁻: m/z=691; 442; 266.

### Example 14

### The Effect of Periodontal Cancer Patients' Saliva on TGL Oxidation

Saliva samples from periodontal cancer patients (n=3) and from healthy subjects (n=5) were collected and immediately centrifuged to remove solid particles. 1.5 ml from the supernatant of each sample were taken into a tube, into which 8 μl of the marker TGL (from a 10 mM stock solution) and 5 μl of internal standards (heptadecanoic acid and 19-hydroxycholesterol from a 5000 ppm stock solution in ethanol) were then added. The mixtures of the saliva together with the standards and the marker were vortex gently and incubated at room temperature overnight. 10 μl of butylated hydroxyanisole (BHA) solution (from a 5000 ppm stock solution) were then added to each tube and, after vortex, 4.5 ml of hexane:2-propanol (3:2 v/v) were added for extracting the marker. After additional vortex, the organic solvents were separated (upper phase), and the extraction procedure was repeated once again with additional 4.5 ml solvents.

The combined organic phases were dried using anhydrous sodium sulfate (0.25 g), vortex, filtered, and the organic solvents were then evaporated by purging nitrogen to dryness. Samples were stored at −20° C. until analysis. Contrary to the analysis of the TCL marker, described in Example 9 hereinabove, the analysis of the oxidized marker units was performed directly, without first hydrolyzing the ester bond between the 2′-deoxyguanosine residue and the tyrosine residue, by injection the entire marker to LC/MS/MS and analyzing simultaneously oxidation in the linoleic acid, tyrosine and guanosine sub-units at the same time. The results are summarized in Table 7, for 3 cancer patients (SC1A, SC1B and SC3) and 5 healthy patients (SN1 to SN5).

The efficiency of the extraction process was determined based on the recovered amount of the internal standards previously added to the mixture. Based on the amount of these standards, it is clear that the procedure and the analytical part were acurate within the level of ±20%. As shown in Table 7, the TGL can detect reactive oxygen species in saliva. However, whereas the linoleic acid and the tyrosine obtained moieties of the marker (TL-OOH and TL-epoxy) seem to be less correlated with observed factors, it seems that the guanosine is more sensitive to oxidation within the cancer group and that the level of oxidized guanosine obtained (8-oxo-guanosine) as a result of the incubation of the marker with saliva samples from periodontal cancer patients is significantly higher than the oxidation level occurs during workup, as known from the prior art (about 0.4%) and further supported by Table 7, with regard to healthy subjects.

TABLE 7
Ox-TL products and 8-oxo-guanosine vs. TL and guanosine
total amounts, respectively, in periodontal cancer disease
patients vs. healthy individuals (in μM)

| | TL-OOH | TL-epoxy | 8-oxo-guanosine |
|---|---|---|---|
| SC1A | 0.571 | 1.246 | 1.651 |
| SC1B | 0.676 | 1.046 | 1.756 |
| SC3 | 0.382 | 0.678 | 2.242 |
| SN1 | 0.187 | 1.085 | 0.505 |
| SN2 | 0.426 | 1.503 | 0.725 |
| SN3 | 0.670 | 1.922 | 0.401 |
| SN4 | 0.176 | 0.525 | 0.415 |
| SN5 | 0.218 | 0.821 | 0.451 |

### Example 15

### TL Distribution 2-18 h Following IV Injection to Mice

A solution of 200 μl of the marker TL (from a stock solution of 100 mM) in DMSO was injected intravenously (tail) to female mice (this amount represents 280 mg/kg body weight, which seems to be the highest non-lethal concentration). After 2, 6 and 18 h in each time interval a mouse was sacrificed and the following organs—blood, liver, kidneys, spleen, heart, lungs, skeletal muscle, brain, ovaries, intestine and pancreas—as well as the urine when possible were removed and collected for homogenization, followed by sonication and extraction.

Each organ was transferred directly to a tube containing 3 ml of a mixture of hexane:2-propanol (3:2 v/v) containing 0.01% BHA. However, in case the organ was too big (liver), additional 1-2 ml of the organic solution were added. The organs in the organic solution were processed on the same day, homogenized in the organic solvents and then sonicated for 3×20 seconds. After vortex and centrifugation (3000 rpm, 3 min), the organic solvents (upper phase) were separated, additional 2-3 ml of the organic solvents were added to the tubes, vortexed, centrifuged and organic solvents separation was performed. The collected organic solvents were dried using anhydrous sodium sulfate (0.2 g), filtered and evaporated under nitrogen purging, and the dried products were kept at −20° C. until shipment. In case urine was available, urea was collected from a paper which was wet and the paper was then extracted directly with the same organic solvents.

Table 8 summarizes the TL distribution in the various organs after 2, 6 and 18 hrs, using arbitrary units based on the area under the peak of each identified oxidized product. This table is aimed to demonstrate the level of the marker in each one of the organs after each period of time and may assist in determining the most relevant organ(s) to be tested for the markers oxidized products and the preferred timing for these examinations. Based on the preliminary findings presented in Table 8, the concentration of the marker in the blood 2 hrs after injection was the highest in comparison with its concentration 6/18 hrs after injection, and is about 52 times higher than its concentration in the liver. Hence, if oxidative stress in blood is under investigation, collecting blood after 2 hrs or shorter time period should be sufficient. The skeletal muscle seems to be the major organ in which TL is accumulating and 6 hrs after injection it contains the highest concentration of the marker, in comparison to the other organs. Additional conclusion resulted from this experiment is that increase of incubation time above 6 hrs does not necessarily result with accumulation of the marker TL in certain organs.

TABLE 8
TL marker distribution 2-18 h following IV injection to mice

| | | |
|---|---|---|
| | | TL units |
| Tissue | Weight (mg) | (area under peak) |

2 hours after IV injection

| | | |
|---|---|---|
| Blood | 217 | 5252 |
| Liver | 742 | 106 |
| Kidneys | 260 | 141 |
| Spleen | 65 | 87 |
| Heart | 103 | 155 |
| Lungs | 91 | 676 |
| Skeletal muscle* | 407 | 950 |
| Brain | 411 | 19 |
| Ovaries | 8 | 0 |
| Pancreas | 153 | 54 |
| Urine | 220 (with paper) | 17 |

6 hours after IV injection

| | | |
|---|---|---|
| Blood | 335 | 186 |
| Liver | 696 | 259 |
| Kidneys | 316 | 191 |
| Spleen | 62 | 110 |
| Heart | 125 | 253 |
| Lungs | 210 | 816 |
| Skeletal muscle* | 357 | 44481 |
| Brain | 944 | 87 |
| Ovaries | 9 | 0 |
| Pancreas | 197 | 162 |
| Urine | 33 (with paper) | 20 |

18 hours after IV injection

| | | |
|---|---|---|
| Blood | 298 | 1526 |
| Liver | 709 | 375 |
| Kidneys | 235 | 117 |
| Spleen | 71 | 219 |
| Heart | 104 | 98 |
| Lungs | 116 | 172 |
| Skeletal muscle* | 453 | 5906 |
| Brain | 419 | 52 |
| Ovaries | 10 | 0 |
| Pancreas | 237 | 346 |
| Urine | — | — |

*Only a piece was removed, collected and treated

### Example 16

### Exogenous Markers TL, TCL and TGL Comparative Distribution 2-6 h Following IV Injection to Mice

The aim of this experiment, being a continuation of the experiment described in Example 15 hereinabove, is to analyze the level of each of the exogenous markers as well as their oxidized products in the various organs (if any distribution selectivity takes place), and the effect of the time period after injection.

A mixture of the three markers with a total volume of 180 μl containing 60 μl of N-linoleyl tyrosine (TL, from a stock solution of 40 mM), 60 μl of N-linoleyl tyrosine cholesteryl ester (TCL, from a stock solution of 40 mM) and 60 μl of N-linoleyl tyrosine 2′-deoxyguanosine ester (TGL, from a stock solution of 20 mM), is injected intravenously (tail) to 3 groups of 3 female mice each. 0.5, 2 and 6 hrs after the injection one of the groups is sacrificed and the internal organs of the mice (blood, liver, kidneys, spleen, heart, lungs, skeletal muscle, brain, ovaries, intestine, pancreas and urine when possible) are removed and collected for homogenization, followed by sonication and extraction as described in Example 15 hereinabove. The level of each marker as well as its oxidized products is analyzed, using the methods described above, and average arbitrary levels of each of the identified products with respect to each of the organs is calculated, as well as additional statistic parameters.

Analysis of the results of this comparative protocol can enable to determine the distribution of each of the exogenous markers in the various organs in correlation with the time period after injection, and the level of each of the oxidized products identified as a reference. Similar experiments can be then conducted using animal models for different pathologies associated with oxidative/nitrosative stress thus obtaining corresponding data regarding the distribution of each of the markers and their oxidized products in the various organs, and the effect of time period after injection, with respect to each pathological condition examined. By integrating all the data obtained it may be possible to determine the preferred marker(s) to be used in order to identify certain oxidative stress-related pathological conditions and the preferred incubation time and organ(s) to be tested, as well as to define a specific oxidative stress fingerprint with respect to each of the pathological conditions examined.

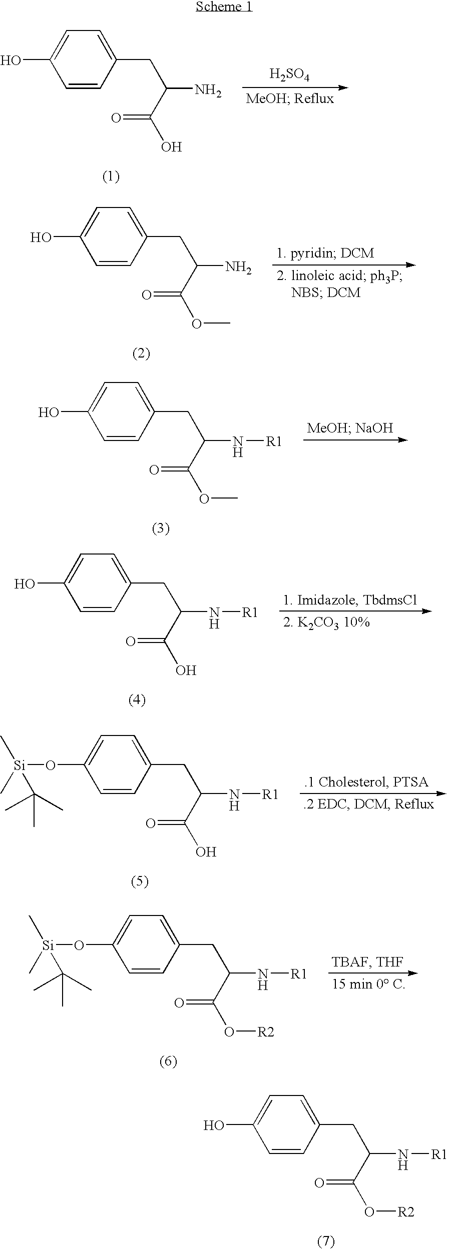

R1=linoleic acid; R2=cholesterol; DCM=dichloromethane; THF=tetrahydrofuran; MeOH=methanol; ph₃P=triphenylphosphine; NBS=N-bromosucciniamide; TbdmsCl=t-butyldimethylsilylchloride; PTSA=p-toluenesulfonic acid; TBAF=tetrabutylamoniumfluoride.

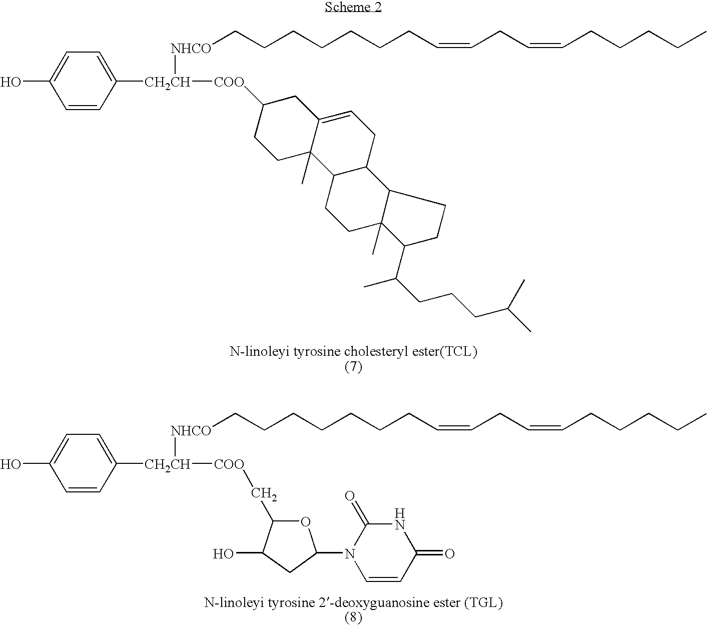

### REFERENCES

Abuja, P. M. Albertini, R. Esterbauer, H., Chem Res Toxicol, 1997, 10, 644-651

Aviram, M., Free Radic Res., 2000, 33, S85-97

Aviram, M. Fuhrman, B., Mol Cell Biochem, 1998, 188, 149-159

Aviram, M. Rosenblat, M. Etzioni, A. Levy, R., Metabolism, 1996, 45, 1069-1079

Aviram, M. Vaya, J., Methods Enzymol, 2001, 335, 244-256

Beckmann, J. S. Ye, Y. Z. Anderson, P. G. Chen, J. Accavitti, M. A. Tarpey, M. M. White, C. R., Biol Chem Hoppe Seyler, 1994, 375, 81-88

Botti, H. Batthyany, C. Trostchansky, A. Radi, R. Freeman, B. A. Rubbo, H., Free Radic Biol Med, 2004, 36, 152-162

Burkitt, M. J., Arch Biochem Biophys, 2001, 394, 117-135

Cadenas, E. Sies, H., Free Radic Res, 1998, 28, 601-609

Chapman, A. L. Senthilmohan, R. Winterbourn, C. C. Kettle, A. J., Arch Biochem Biophys, 2000, 377, 95-100

Eiserich, J. P. Hristova, M. Cross, C. E. Jones, A. D. Freeman, B. A. Halliwell, B. van der Vliet, A., Nature, 1998, 391, 393-397

Finkel, T. Holbrook, N. J., Nature, 2000, 408, 239-247

Froyen, P., Tetrahedron Letters, 1997, 38, 5359-5362

Fuhrman, B. Shiner, M. Volkova, N. Aviram, M., Free Radic Biol Med, 2004, 37, 259-271

Halliwell, B., Cardiovasc Res, 2000, 47, 410-418

Halliwell, B., Free Radic Biol Med, 2002, 32, 968-974

Halliwell, B. Gutteridge, J. M. C., Free radicals in biology and medicine, 1999, 3^{rd} ed., Oxford University Press, Oxford

Hayek, T. Oiknine, J. Brook, J. G. Aviram, M., Biochem Biophys Res Commun, 1994, 201, 1567-1574

Hazell, L. J. van den Berg, J. J. Stocker, R., Biochem, 1994, J302, 297-304

Hazen, S. L. Heinecke, J. W., J Clin Invest, 1997, 99, 2075-2081

Heller, J. I. Crowley, J. R. Hazen, S. L. Salvay, D. M. Wagner, P. Pennathur, S. Heinecke, J. W., J Biol Chem, 2000, 275, 9957-9962

Jerlich, A. Pitt, A. R. Schaur, R. J. Spickett, C. M., Free Radic Biol Med, 2000, 28, 673-682

Lynch, S. M. Frei, B., J Biol Chem, 1995, 270, 5158-5163

Maor, I. Kaplan, M. Hayek, T. Vaya, J. Hoffman, A. Aviram, M., Biochem Biophys Res Commun, 2000, 269, 775-780

Moore, K. P. Darley-Usmar, V. Morrow, J. D. Roberts, L. J., Circ Res, 1995, 77, 335-341

Plump, A. S. Smith, J. D. Hayek, T. Aalto-Setala, K. Walsh, A. Verstuyft, J. G. Rubin, E. M. Breslow, J. L., Cell, 1992, 71, 343-353

Nunoi, H. Rotrosen, D. Gallin, J. I. Malech, H. L., Science, 1988, 242, 1298-1301

Radi, R., Proc Natl Acad Sci USA, 2004, 101, 4003-4008

Rosenblat, M. Aviram, M., Atherosclerosis, 2002, 160, 69-80

Saran, M. Beck-Speier, I. Fellerhoff, B. Bauer, G., Free Radic Biol Med, 1999, 26, 482-490

Szuchman, A. Aviram, M. Tamir, S. Vaya, J., Free Radic Res, 2003, 37, 1277-1288

Vaya, J. Aviram, M. Mahmood, S. Hayek, T. Grenadir, E. Hoffman, A. Milo, S., Free Radic Res, 2000, 34, 485-497

1. A compound of the formula I: 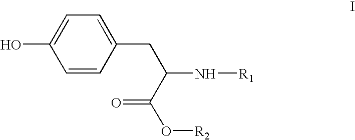
wherein R₁ is linoleyl, and R₂ is cholesteryl or a 2′-deoxyguanosine residue.

2. (canceled)

3. The compound according to claim 1, wherein R₂ is cholesteryl.

4. The compound according to claim 1, wherein R₂ is 2′-deoxyguanosine.

5. A marker for oxidative stress consisting of a compound of formula I in claim 1, wherein R₁ is linoleyl, R₂ is H, cholesteryl or a 2′-deoxyguanosine residue, and oxidation products of said marker increase in a biological sample compared to a control sample during oxidative stress.

6. A method of assessing oxidative stress which comprises incubation of a biological sample with a marker of formula I in claim 1, wherein R₁ is linoleyl, and R₂ is H, cholesteryl or a 2′-deoxyguanosine residue, whereby an increase of the amount of at least one oxidation product of said marker in the biological sample compared to a control sample indicates oxidative stress.

7. The method of claim 6, wherein the marker is the compound of formula I wherein R₂ is H.

8. The method of claim 6, wherein the marker is the compound of formula I wherein R₂ is cholesteryl.

9. The method of claim 6, wherein the marker is the compound of formula I wherein R₂ is a 2-deoxyguanosine residue.

10. The method of claim 6, wherein the biological sample is blood, urine or saliva sample.

11-13. (canceled)
